(19) United States
(12) Patent Application Publication
(10) Pub. No.: US 2024/0132430 A1
(43) Pub. Date: Apr. 25, 2024

(54) CHEMICALS AND USE OF HYPOHALITES IN MECHANISM-BASED SELECTIVE DUAL RADICAL ORGANIC SYNTHESIS

(71) Applicant: WALKER CANCER RESEARCH INSTITUTE, INC., Boynton Beach, FL (US)

(72) Inventor: Eduardo PALOMINO, Troy, MI (US)

(73) Assignee: WALKER CANCER RESEARCH INSTITUTE, INC., Boynton Beach, FL (US)

(21) Appl. No.: 18/390,314

(22) Filed: Dec. 20, 2023

Related U.S. Application Data
(63) Continuation-in-part of application No. 17/394,680, filed on Aug. 5, 2021.

(51) Int. Cl. C07C 37/88; C07B 41/02; C07C 37/06

(52) U.S. Cl. CPC C07C 37/88; C07B 41/02; C07C 37/06

(57) ABSTRACT

The synthesis of pattern-specific compounds using hypohalites, such as hypochlorous acid, sodium hypochlorite, and potassium hypoiodite, as dual-radical generators is provided. The synthesis can be implemented by a cyclization reaction, a dehydrogenation reaction, a hydroxylation reaction, a decarboxylation reaction, or any combination of the above four. The reactions are typically carried out in water, in a nonpolar solvent such as ethyl acetate, or a mixture of both. Hypochlorous acid is made by adding a weak acid such as acetic acid to sodium hypochlorite.

### CROSS-REFERENCE TO RELATED APPLICATIONS

This application is a continuation-in-part of U.S. application Ser. No. 17/394,680 filed Aug. 5, 2021, the disclosure of which is hereby incorporated in its entirety by reference herein.

### FIELD OF DISCLOSURE

In at least one aspect, the present invention relates the use of hypohalites in mechanism-based selective dual-radical organic syntheses.

### BACKGROUND OF THE INVENTION

The use of radicals in organic syntheses is abundant in the industry and employs mostly toxic materials that generate harmful by-products

Accordingly, there is a need for safe and effective radical organic syntheses devoid of dangerous by-products.

### SUMMARY OF THE INVENTION

In at least one aspect, the present invention provides an exemplary use of hypohalites, such as hypochlorous acid, sodium hypochlorite and potassium hypoiodite, as dual-radical reagents for the synthesis of pattern-specific-compounds. In this regard, unlike most synthetic methodologies that use one reagent to produce one chemical action, the method employed in the present invention targets two selective sites of a starting material using the dual-radical reagent to achieve the overall reaction. According to an embodiment, the dual-radical reagent is sequential in nature: first, the hypohalite radical removes a hydrogen atom from an appropriate donor generating hydroxyl radical as the second part of the radical system , where formation of the hydroxyl radical hinges entirely on the reaction of the hypohalite radical. As such, only those compounds containing the correct pattern in the starting material can react. Such reactions will generally be safe and produce no toxic compounds. For example, sodium chloride and water are the usual by-products.

In another aspect, the following chemical transformations with pattern-specific starting materials can be achieved with the exemplary process:
a. Cyclization reactions of compounds containing a phenol group and an amino group. Examples include the synthesis of the known dopaminochrome and indole.
b. Dehydrogenation reactions of compounds containing two phenol groups in the same ring, or a phenol and an amino group in the same ring. Examples include the synthesis of the known 1,2 and 1,4 benzoquinones and 1,2 and 1,4 iminoquinones.
c. Hydroxylation reactions of compounds containing a phenol group and a hydrogen on an adjacent carbon to the phenol (ortho position) or two carbons apart (para position). Examples include the synthesis of the unreported tococatechols of 6-tocopherol and the known E-quinone from α-tocopherol.
d. Decarboxylation reactions, with or without cyclization, of starting materials containing an amino group and a carboxyl (acid) group attached to the same carbon. Examples include the synthesis of the known compounds indole and 1-pyrroline.
e. Trichlorination reactions of methyl ketones in a single step. Examples include the synthesis of highly priced trichloroacetone and trichloroacetophenone and the unreported tococatechols.

### BRIEF DESCRIPTION OF THE DRAWINGS

Some aspects of the disclosure are herein described, by way of example only, with reference to the accompanying drawings. With specific reference now to the drawings in detail, it is stressed that the particulars shown are by way of example and are for purposes of illustrative discussion of embodiments of the disclosure. In this regard, the description, taken with the drawings, makes apparent to those skilled in the art how aspects of the disclosure may be practiced.

FIG. 1 illustrates a decomposition of hydrogen peroxide by the enzyme catalase.

FIG. 2 illustrates a mechanism of reaction of the hydroxyl radical generator K₃HFe(CN)₆ with hydrogen peroxide, and quenching of the hydroxyl radical.

FIG. 3 illustrates the formation of a 1,4-iminoquinone by the action of potassium ferricyanide on p-aminophenol.

FIG. 4 illustrates a mechanism of reaction of an enzyme catalase on hydrogen peroxide through the formation of a hydroxyl radical intermediate.

FIG. 5 illustrates a formation of 1,4-iminoquinone from p-aminophenol by the hydroxyl radical produced in the reaction of catalase with hydrogen peroxide.

FIG. 6 illustrates the formation of dopaminochrome from dopamine by the hydroxyl radical produced in the reaction of catalase with hydrogen peroxide.

FIG. 7 illustrates the mechanism of the formation of dopaminochrome from dopamine via the reaction of catalase with hydrogen peroxide.

FIG. 8 illustrates the reaction of hydrogen peroxide with sodium hypochlorite to produce molecular oxygen.

FIG. 9 illustrates the reaction of hydroquinone with sodium hypochlorite to produce p-benzoquinone via a dehydrogenation reaction.

FIG. 10 illustrates the mechanism of the reaction of hydroquinone with sodium hypochlorite of FIG. 9 that demonstrates the dual-radical character of hypochlorite.

FIG. 11 illustrates a mechanism of the reaction of hydrogen peroxide with sodium hypochlorite via the dual-radical character of hypochlorite.

FIG. 12 illustrates the mechanism of the reaction of 1,4-aminophenol with sodium hypochlorite to produce 1,4-iminoquinone and demonstrates the dual-radical character of hypochlorite.

FIG. 13 illustrates the mechanism of the formation of dopaminochrome by reaction of two units of sodium hypochlorite with one unit of dopamine via the dual-radical system of hypochlorite.

FIG. 14 illustrates the mechanism of the reaction of α-tocopherol with sodium hypochlorite to produce E-quinone via the dual-radical system of hypochlorite. R is a C₁₋₂₂ branched or linear alkyl.

FIG. 15 illustrates the mechanism of the formation of the two isomers of E-catechol from δ-tocopherol (a vitamin E) with sodium hypochlorite via the dual-radical system of hypochlorite. R is a C₁₋₂₂ branched or linear alkyl.

FIG. 16 illustrates the mechanism of the formation of indole and 1-pyrroline by decarboxylation with sodium hypochlorite via the dual-radical system of hypochlorite.

FIG. 17 illustrates the reaction of potassium iodide with hydrogen peroxide to produce potassium hypoiodite and the reactions of the potassium hypoiodite with hydrogen peroxide to produce molecular oxygen and with 1,4-aminophenol to produce 1,4-iminoquinone via the dual-radical system of the hypoiodite.

FIG. 18 illustrates a general diagram showing the basic series of events in reactions using sodium hypochlorite.

FIG. 19 illustrates a method for making p-quinones via a dehydrogenation reaction.

FIG. 20 illustrates a method for making aminochromes via a cyclization reaction.

FIG. 21 illustrates a method for making catechols via a hydroxylation reaction.

FIG. 22 illustrates a method for making p-imino quinone via a dehydrogenation reaction.

FIG. 23 illustrates a method for making trichloromethyl ketones.

FIG. 24 illustrates a method for making a dehydroascorbic acid via a dehydrogenation reaction.

FIG. 25 provides the crystal structure for trolox quinone.

### DETAILED DESCRIPTION OF THE INVENTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: all R groups (e.g. Rᵢ where i is an integer) include hydrogen, alkyl, lower alkyl, C₁₋₆ alkyl, C₆₋₁₀ aryl, C₆₋₁₀ heteroaryl, —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁺, —COO⁻M⁺, —CF₂H, —CF₂R′, —CFH₂, and —CFR′R″ where R′, R″ and R′″ are C₁₋₁₀ alkyl or C₆₋₁₈ aryl groups M is a metal atom (e.g., Na, K, Li, etc.) and L− is a counter anion (e.g., Cl−, Br−, tosylate, etc.); single letters (e.g., “n” or “o”) are 1, 2, 3, 4, or 5; in the compounds disclosed herein including compounds described by formula or by name, a CH bond can be substituted with alkyl, lower alkyl, C₁₋₆ alkyl, C₁₋₂₂ linear or branched alkyl, C₆₋₁₀ aryl, C₆₋₁₀ heteroaryl, —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁺, —COO⁻M⁺, —CF₂H, —CF₂R′, —CFH₂, and —CFR′R″ where R′, R″ and R′″ are C₁₋₁₀ alkyl or C₆₋₁₈ aryl groups where M is a metal atom (e.g., Na, K, Li, etc.) and L− is a negative counterion (e.g., Cl⁻, Br⁻, etc.); percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

The term “alkyl” refers to C₁₋₂₂ inclusive, linear (i.e., “straight-chain”), branched, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 9 carbon atoms (i.e., a C₁₋₈ alkyl), e.g., 1, 2, 3, 4, 5, 6, 7, 8, or 9 carbon atoms. “Higher alkyl” refers to an alkyl group having about 10 to about 22 carbon atoms, e.g., 10, 11, 12, 13, 14, 15, 16, 17, 18, 19, 20, 21, or 22 carbon atoms.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

The phrase “composed of” means “including” or “comprising.” Typically, this phrase is used to denote that an object is formed from a material.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits. In the specific examples set forth herein, concentrations, temperature, and reaction conditions (e.g. pressure, pH, etc.) can be practiced with plus or minus 50 percent of the values indicated rounded to three significant figures. In a refinement, concentrations, temperature, and reaction conditions (e.g., pressure, pH, etc.) can be practiced with plus or minus 30 percent of the values indicated rounded to three significant figures of the value provided in the examples. In another refinement, concentrations, temperature, and reaction conditions (e.g., pH, etc.) can be practiced with plus or minus 10 percent of the values indicated rounded to three significant figures of the value provided in the examples.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

The term “weak organic acid” refers to a carboxylic acid, dicarboxylic acid, or tricarboxylic acid having a pKa from 2 to 7.5. In a refinement, refers to a carboxylic acid, dicarboxylic acid, or tricarboxylic acid having a pKₐ of at least 2, 2.5, 3, 3.5, or 4 and of at most 7.5, 7, 6.5, 6, 5.5 or 5. Examples of weak organic acids include but are not limited to, acetic acid, formic acid, propionic acid, butyric acid, benzoic acid, lactic acid, and oxalic acid. It is noteworthy that acetic acid has a pKₐ of about 4.76.

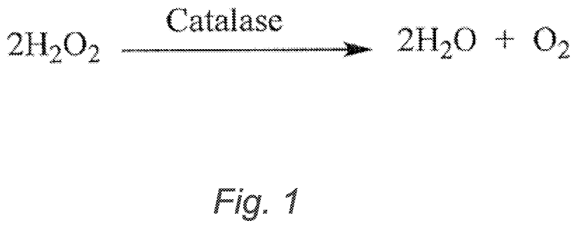

In at least one aspect, one of the most abundant radicals in the animal environment is the hydroxyl radical (OH), produced naturally by enzymes but also by the interplay of compounds such as ferric iron (Fe⁺³) and ascorbic acid (vitamin C). The hydroxyl radical produced by nature is used as a tool to perform essential reactions, it also serves as a signaling molecule of vital conditions and to destroy invading attackers such as viruses and bacteria. It was found that the enzyme catalase, present extensively in the human body to perform the conversion of hydrogen peroxide (H₂O₂) to molecular oxygen (O₂) and water (H₂O) as shown as the overall reaction in FIG. 1, generates the hydroxyl radical as an intermediate

In another aspect, by using the reported crystal structures of the enzyme and the hydroxyl radical generator K₃HFe(CN)₆, which also converts H₂O₂ to O₂ and H₂O but through the formation of OH radicals as shown in 21 2, the mechanism of action of catalase has been elucidated.

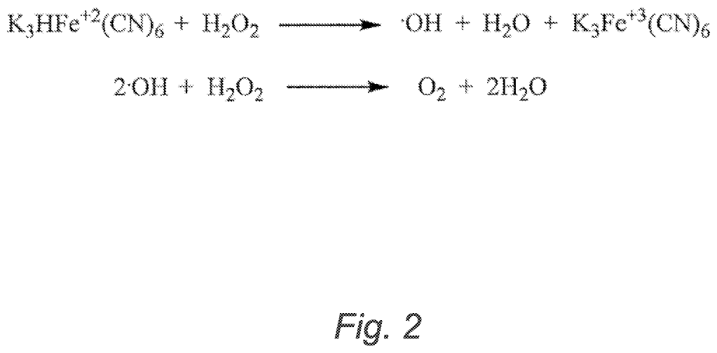

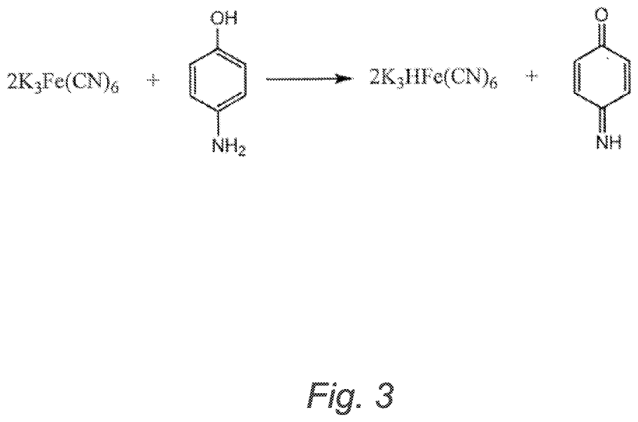

In another aspect, the catalase enzyme has in its native state a hydrogenated heme iron (heme-H) in a low oxidation state in close proximity to the oxygen of the phenol tyrosine that is actually a tyrosyl radical. Similar to the synthesis of K₃HFe(CN)₆ shown in FIG. 3, catalase uses the equivalent reactions of FIG. 2 and FIG. 3 to achieve a similar process in a catalytic fashion.

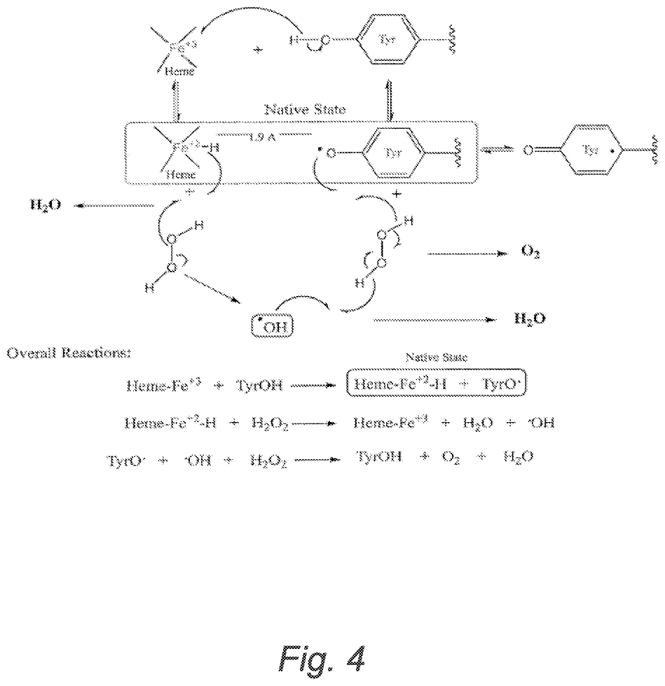

In another aspect, the mechanism of catalase is shown in FIG. 4 where the reduced heme-H reacts with H₂O₂ to produce oxidized heme, one OH radical plus water. The tyrosyl radical participates with the OH radical to remove hydrogens from the peroxide to produce molecular oxygen and a tyrosine molecule that repeats the cycle by reacting with the oxidized heme.

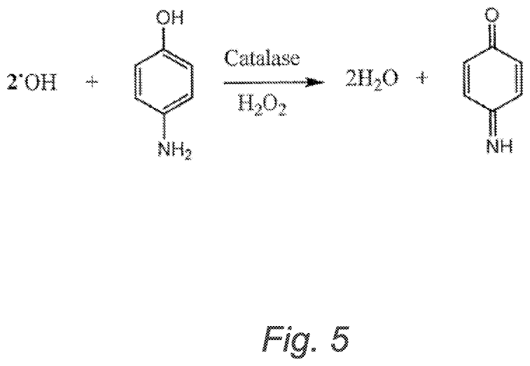

In another aspect, to demonstrate the production and involvement of the hydroxyl radical in the catalase mechanism, the same reaction of FIG. 3 with 1,4-aminophenol was performed producing the red 1,4-iminoquinone as shown in FIG. 5.

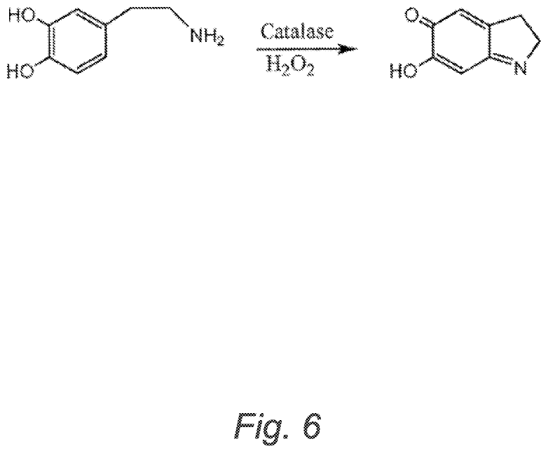

Furthermore, a reaction similar to the one displayed in FIG. 5 can be obtained using dopamine hydrochloride, a water-soluble catecholamine involved in numerous processes in the human body. When dopamine hydrochloride is added to the catalase reaction, the strongly red dopaminochrome is produced as shown in FIG. 6.

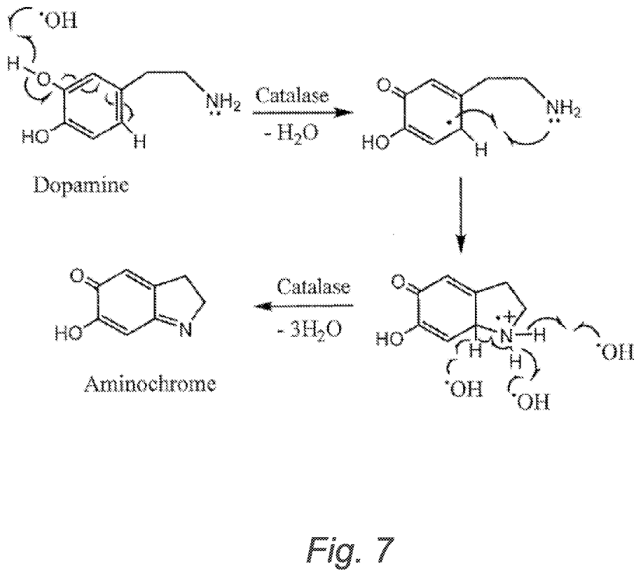

In another aspect, despite the fact that FIGS. 3, 5, and 6 show the involvement of an amino (NH₂) group, the hydroxyl radical is unable to attack the NH₂ hydrogens directly as demonstrated by its lack of reactivity with aliphatic and aromatic amines. The hydroxyl radical exclusively attacks phenolic hydrogens and the OH group of vitamin C, thus, the mechanism of FIGS. 3, 5, and 6 are explained by the contribution of electrons from the NH₂ group to a preformed aromatic radical as shown for dopamine in FIG. 7.

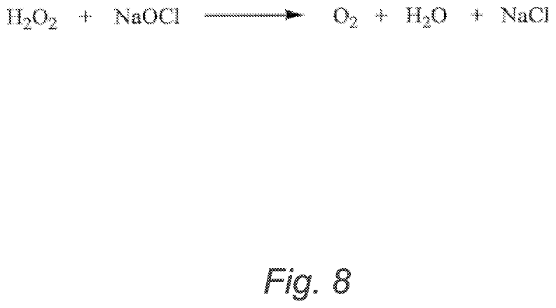

In another aspect, the importance of the result of FIG. 7 lies on the fact that dopaminochrome is the chemical involved in serious neurologic illnesses such as Parkinson and Alzheimer diseases, and the demonstration here of its simple formation through hydroxyl radicals has serious health issues. On that vein, other natural sources of hydroxyl radicals were sought and a forgotten published report of the radicals being formed from the reaction of sodium hypochlorite (NaOCl) with hydrogen peroxide shown in FIG. 8 was of interest because the hypochlorite ion (⁻OCl) is an abundant defense product of the immune system, formed in neutrophils, monocytes and many other cells by the ubiquitous enzyme myeloperoxidase (MPO).

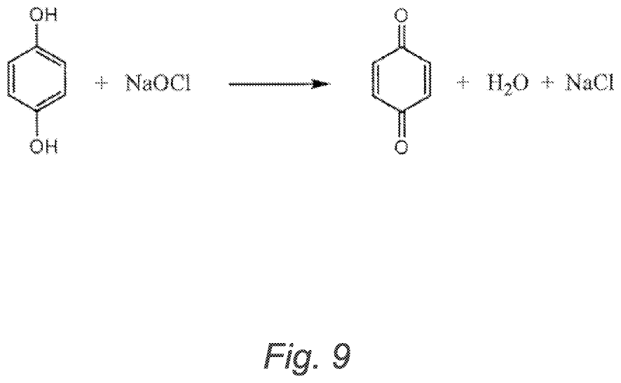

In another aspect, the reaction of FIG. 8 seems simple enough so the formation of the reported hydroxyl radicals represents a puzzle worth pursuing. With the knowledge acquired with the hydroxyl generator, we addressed the problem by reacting hypochlorite with hydroquinone, a hydrogen peroxide equivalent, as shown in FIG. 9. The reaction performed in an alcohol/water mixture is clean and fast producing the known 1,4-benzoquinone in 100% yield using a 1:1 ratio of the reagents.

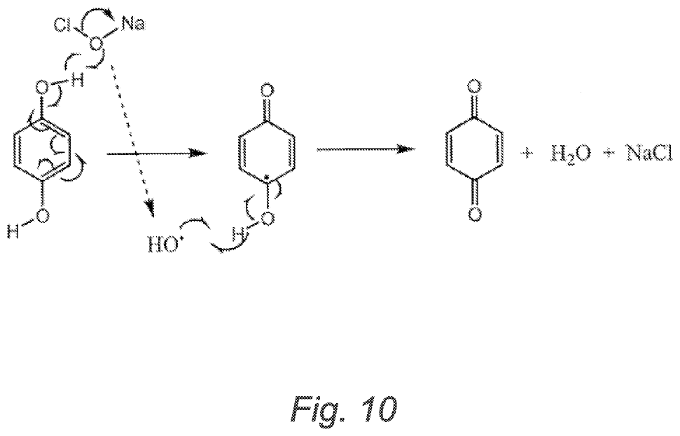

Furthermore, since the reaction of FIG. 9 proceeds equally well with two units of hydroxyl radicals, it implies that two radicals are “contained” within the NaOCl molecule and one of them should be the reported hydroxyl radical that can only be formed after removal of a hydrogen from the hydroquinone by a hypochlorite radical, as shown in FIG. 10.

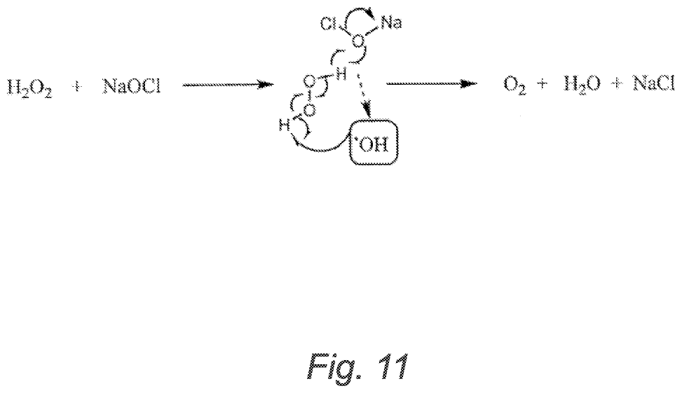

In another aspect, the reaction of FIG. 10 facilitates the elucidation of the mechanism of hypochlorite with hydrogen peroxide as shown in FIG. 11

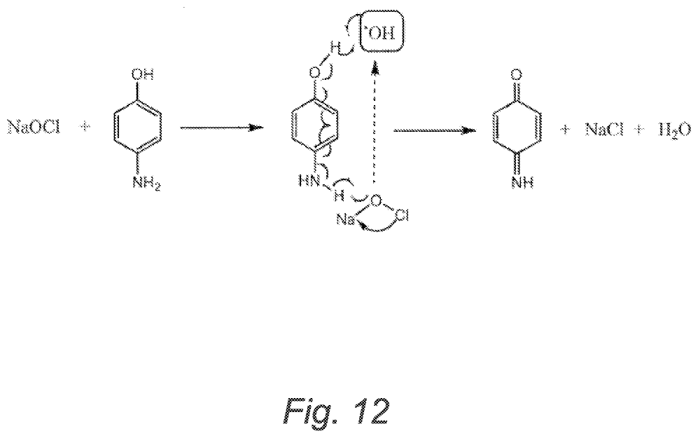

Furthermore, because the reaction is not concerted, the hydroxyl radicals can be observed with ease especially when the concentration of peroxide decreases. Application of the discovered dual radical character of hypochlorite to the reaction of 1,4-aminophenol can be done in a biphasic system of ethyl acetate/water, a method that simplifies purification of the 1,4-iminoquinone of FIG. 5. However, the mechanism here is sidelined by the strong known attraction between hypochlorite and the amino groups, which in simple situations produces chloramines, so the first step of the mechanism is the removal of the amino hydrogen by the hypochlorite radical, as shown in FIG. 12.

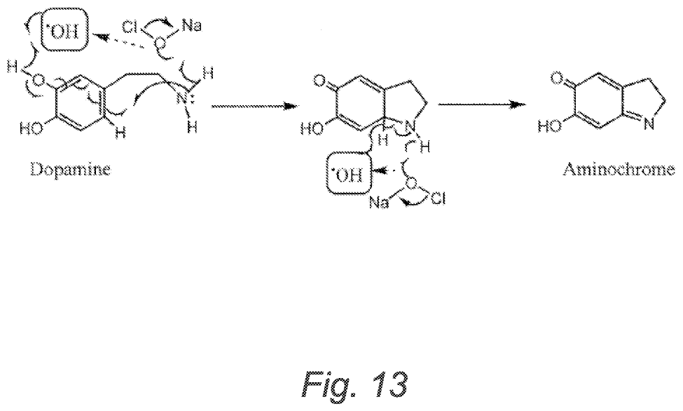

Furthermore, the formation of dopaminochrome proceeds expeditiously with only two units of hypochlorite as opposed to the 4 units of hydroxyl radicals required with catalase, as shown in FIG. 13.

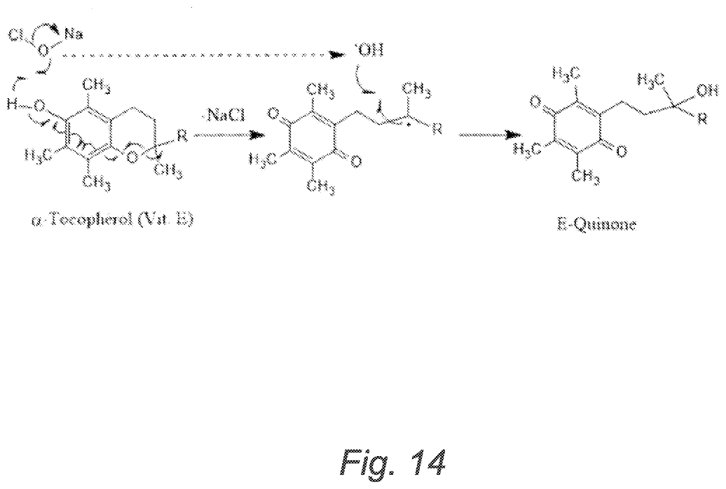

In another aspect, the new discovery of the dual-radical character of hypochlorite in which a hydroxyl radical is involved, ushered a search for the synthesis of compounds that are typically difficult or unattainable by other methods. In that vein, E-quinone, the metabolite of α-tocopherol, one of four vitamin E, which have been successfully produced using the hydroxyl radical was evaluated with this simple method. The reaction of α-tocopherol with one equivalent of hypochlorite in an ethanol/water mixture produces 100% yield of pure red E-quinone with sodium chloride (NaCl) as the only by-product. Following the mechanism outlined before, this synthesis is detailed in FIG. 14 where R is a C₁₋₂₂ branched or linear alkyl.

In another aspect, the importance of an easy and inexpensive synthesis of E-quinone lies in the fact that it allows the study of its involvement in the long known toxicity/interaction with vitamin K, a close structural analog. It has been proposed that the α-tocopherol has toxicity not shared by the β, δ and γ analogues, so the hypochlorite synthesis, a possible replication of a natural process, was used to process the 6-analogue, a compound that differs from the α-analogue in the absence of two methyl (CH₃) groups flanking the OH phenol. Since radical reactions in the aromatic ring follow the electronic rules for substitution, this reaction presents the possibility of explaining the differences in toxicity of the 4 analogues.

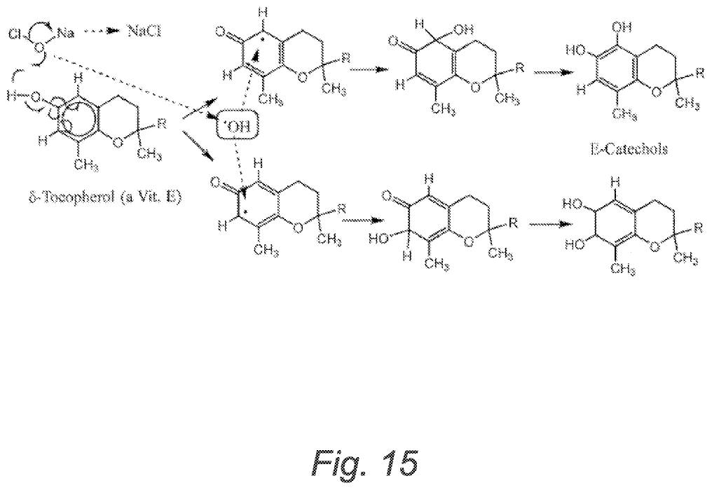

In another aspect, as shown in FIG. 15 where R is a C₁₋₂₂ branched or linear alkyl, attack by the hypochlorite radical can generate two tocopheryl radicals each flanking the initial OH phenol (ortho positions) in addition to the one produced with the α-analogue. When the reaction is run under the same conditions as with α-tocopherol, two compounds are produced in a 1:1 ratio corresponding to the ortho isomers, named tococatechols, with no indication of a quinone formation. This result validates the clinical conclusions that the α-analogue represents an inhibitor of vitamin K.

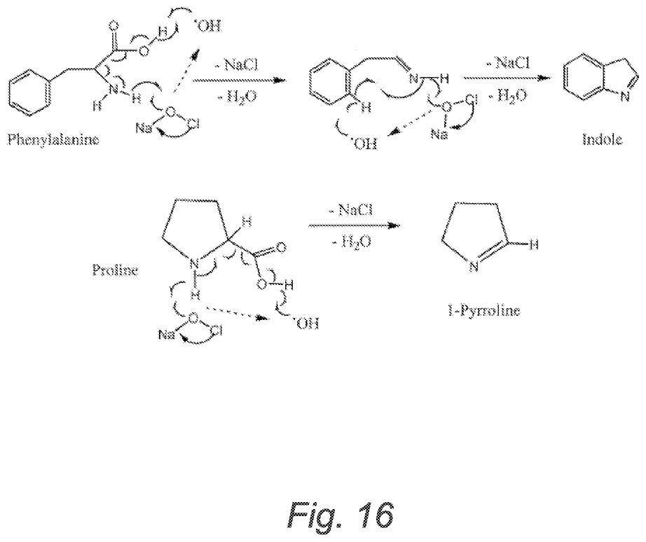

In another aspect, the formation of 1,4-iminoquinone and 1,4-benzoquinone represent new dehydrogenations reactions, dopaminochrome synthesis from dopamine represents a cyclization reaction, whereas E-quinone and tococatechol production are examples of new hydroxylation reactions achieved using the dual-radical character of the hypochlorite, subject of the current application. The areas of activity of this dual radical extend besides those three general methods. When phenylalanine and proline are mixed separately with hypochlorite, the known fecal metabolite indole and the semen-smelling 1-pyrroline are formed, respectively as shown in FIG. 16, in yields larger than 80%, representing yet a new method of decarboxylation with or without cyclization.

Furthermore, the simplicity of the syntheses of FIG. 16 contrasts with the existing current methods of preparation. For 1-pyrroline, for instance, one method uses 2.5 equivalents of the toxic and expensive Sml₂ to cleave the N—O bond of an oxime, and in another, SnCl₄ is employed to promote a cycloaddition between cyclopropanes and nitriles.

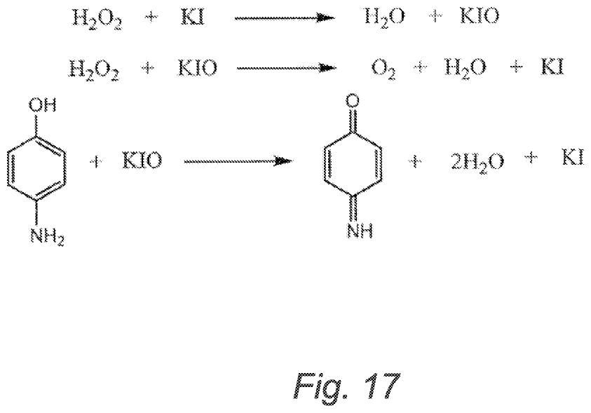

In another aspect, the examples given use sodium hypochlorite as the hypohalite of choice due to ample availability, simplicity of product purification, and low cost. Hypochlorous acid, obtained by acidification of sodium hypochlorite typically to a pH of 3-4 is used to produce materials that are more stable as salts such as the hydrochloride of dopaminochrome, and the hypoiodite can also be employed to perform the same reactions, but, because the hypoiodite is unstable, it is generated in the reaction vessel from the reaction between potassium iodide and hydrogen peroxide. Although hypoiodite can be used at any concentration even in catalytic amounts, the drawbacks for its use is the price of the potassium iodide compared to the hypochlorite and the price of purification of the final product due to the fact that iodine (I₂) is a by-product in this reaction, and iodine gives a yellow color to final materials. The hypoiodite is generated from the reaction of potassium iodide (KI) with hydrogen peroxide and its catalytic use stems from the fact that after reaction with the hydrogen donors, regenerates the iodide starting material, so the only consumable component is the hydrogen peroxide. As shown in FIG. 17, donors include hydrogen peroxide as well as the materials already shown for the hypochlorite.

In another aspect, the requirements for the dual-radical formation process make the reactions: a) very selective, b) prevent the formation of secondary products, and c) releases non-toxic by-products, namely salt and water. Further, the reactions can be completed (with purified products) relatively quickly, making the processes very cost-effective.

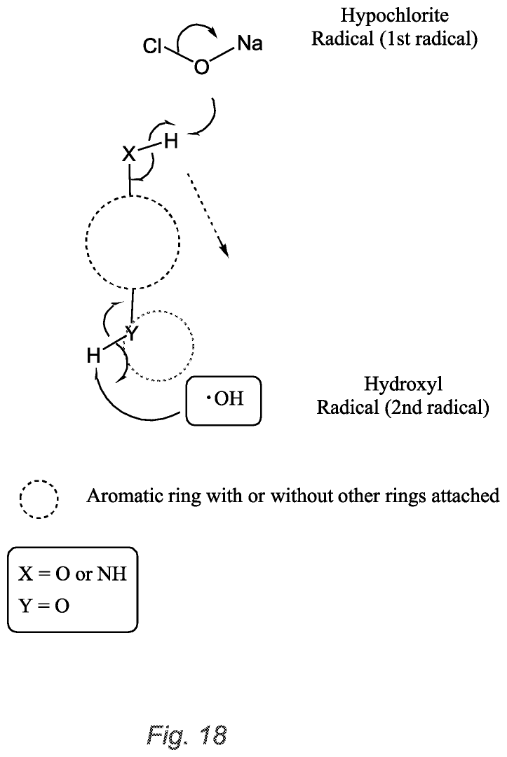

In another aspect, five types of general reactions are covered by this dual radical method in which the basic series of events are outlined in the general diagram of FIG. 18 using sodium hypochlorite as an example. This mechanism is relevant to the following types of reactions:
1. Cyclization reactions of compounds containing a phenol group and an amino group. Examples include the synthesis of dopaminochrome and indole.
2. Dehydrogenation reactions of compounds containing two phenol groups in the same ring, or a phenol and an amino group in the same ring. Examples include the synthesis of quinones and iminoquinones.
3. Hydroxylation reactions of compounds containing a phenol group and a hydrogen on an adjacent carbon to the phenol (ortho position) or two carbons apart (para position). In some ring systems containing a free phenol and substituted phenol in the para position, hydroxylation will occur outside the phenol ring when the substituted ring is opened up generating a radical. Examples include the syntheses of tococatechols and E-quinone.
4. Decarboxylation reactions, with or without cyclization, of compounds containing an amino group and a carboxyl (acid) group attached to the same carbon. Examples include the synthesis of indole and 1-pyrroline.
5. Trichlorination reactions of methyl ketones in a single step. Examples include the synthesis of highly priced trichloroacetone and trichloroacetophenone.

The mechanistic diagram of FIG. 18 can be summarized by a method including a step of reacting a starting compound with hypochlorous acid in an aqueous solution to produce a product compound, the starting compound is described by the following formula:

H—X—R_{B}—R_{A}—R_{C}—OH

wherein:

R_{A} is an optionally substituted aromatic ring, or an optionally substituted aromatic ring fused to a second ring structure, wherein R_{A} is optionally substituted with one or more C₁₋₂₀ alkyl, hydroxyl, or C₁₋₂₀ alkoxy groups;

R_{B} is absent, a C₁₋₂₀ alkyl group or a carboxyl group;

R_{C} is absent, a C₁₋₂₀ alkyl group or a carboxyl group; and

X is absent, O or NH. As set forth above, the starting compound is cyclized when the starting compound includes a phenol group and an amino group; the starting compound is dehydrogenated when the starting compound includes two phenol groups in the same ring or when a phenol and amino group in the same ring; the starting compound hydroxylated when the starting compound described includes a phenol group and a hydrogen on an adjacent carbon to the phenol (ortho position) or two carbons apart (para position); or the starting compound is decarboxylated when the starting compound includes an amino group and a carboxyl (acid) group attached to the same carbon.

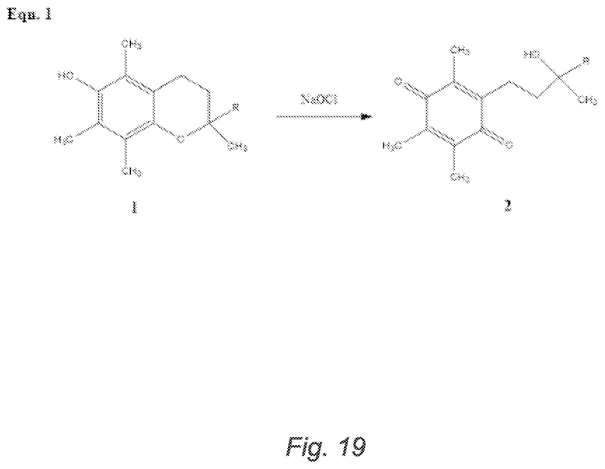

In another aspect, a method for making p-quinones via a dehydrogenation reaction as depicted in FIG. 19 is provided. The method includes a step of forming a biphasic mixture including an aqueous phase that includes a hypochlorite salt and an organic phase that includes a nonpolar solvent, and in particular, an aprotic nonpolar solvent, and a 5,7,8-trimethyl-6-chromanol described by formula 1. Examples of hypochlorite salts include NaOCl, KOCl, LiOCl, and Ca(OCl)₂. The biphasic mixture is agitated (e.g., by shaking, mechanical mixing, and the like) to allow a reaction between the hypochlorite salt with a compound described by formula 1. The organic phase is collected and then the nonpolar solvent is removed to recover a p-quinones described by formula 2:

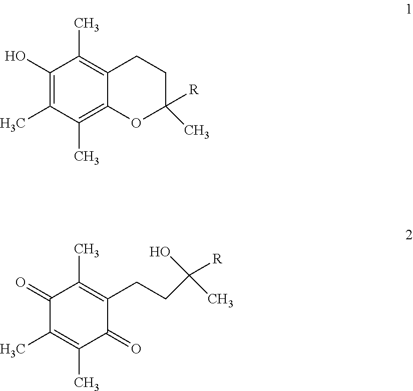

wherein R is selected from linear or branched C₁₋₂₂ alkyl, HC═O, or carboxy. In a refinement, the molar ratio of the hypochlorite salts to the 5,7,8-trimethyl-6-chromanol is about 1:1.

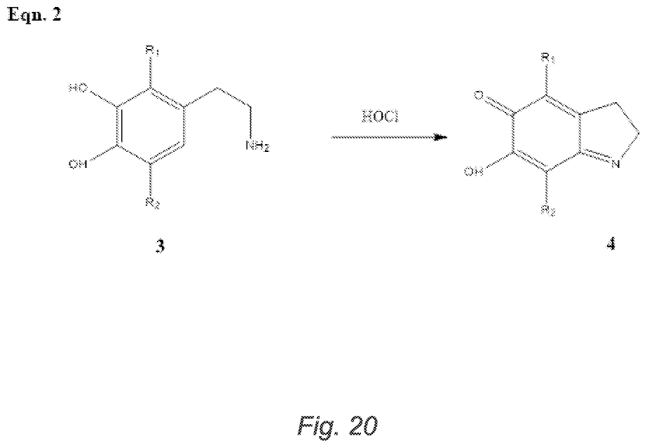

In another aspect, a method for making aminochromes via a cyclization reaction as depicted in FIG. 20 is provided. The method includes a step of combining an aqueous solution that includes a hypochlorite salt with a nonpolar solvent to form a biphasic mixture including an organic phase and an aqueous phase. Examples of hypochlorite salts include NaOCl, KOCl, LiOCl, and Ca(OCl)₂. A weak organic acid is added to the biphasic mixture to form hypochlorous acid. The biphasic mixture is agitated (e.g., by shaking, mechanical mixing, and the like) such that at least a portion of the hypochlorous acid is transferred from the aqueous phase to the organic phase. The aqueous phase is discarded. Therefore, the organic phase is collected. A catecholamine described by formula 3 (typically a solid) is added to the organic phase. The mixture is then agitated until all of the catecholamine is incorporated into the organic phase. The nonpolar solvent is removed to recover an aminochrome described by formula 4:

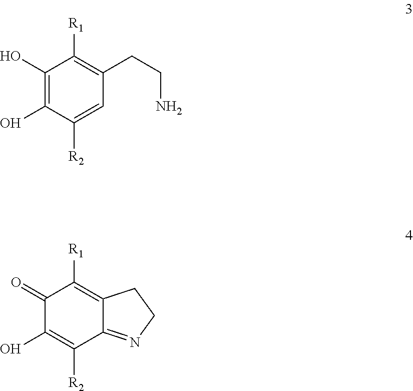

wherein R₁, R₂ are each independently H, alkyl, or alkoxy. In a refinement, the molar ratio of the hypochlorite salt or the hypochlorous acid to the catecholamine described by formula 3 is about 1:1.

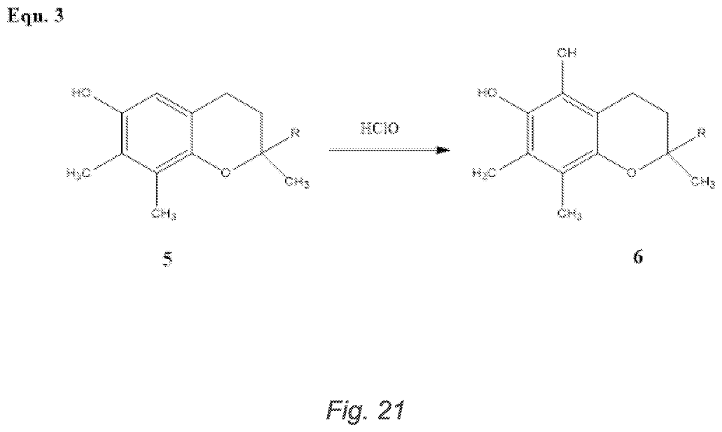

In another aspect, a method for making catechols via a hydroxylation reaction as depicted in FIG. 21 is provided. The method includes a step of forming a biphasic mixture including an aqueous phase that includes a hypochlorite salt and an organic phase that includes a nonpolar solvent, and in particular, an aprotic nonpolar solvent. Examples of hypochlorite salts include NaOCl, KOCl, LiOCl, and Ca(OCl)₂. A weak organic acid (e.g., acetic acid) is added to the biphasic mixture to form hypochlorous acid. The biphasic mixture is agitated to transfer the hypochlorous acid formed in the aqueous phase into the organic phase. The aqueous phase is discarded. Therefore, the organic phase is collected. A chromanol described by formula 5 is added to the organic phase. The mixture is agitated until an acidic aqueous phase is formed. Characteristically, the acidic aqueous phase includes hydrochloric acid (HCl). The acidic aqueous phase is subsequently discarded. The nonpolar solvent is removed and then a catechol having the formula 6 is collected:

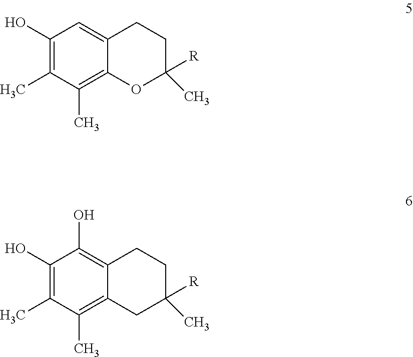

wherein R is selected from linear or branched C₁₋₂₂ alkyl, HC═O, or carboxy. In a refinement, the molar ratio of the hypochlorite salt or the hypochlorous acid to the chromanol described by formula 5 is about 1:1.

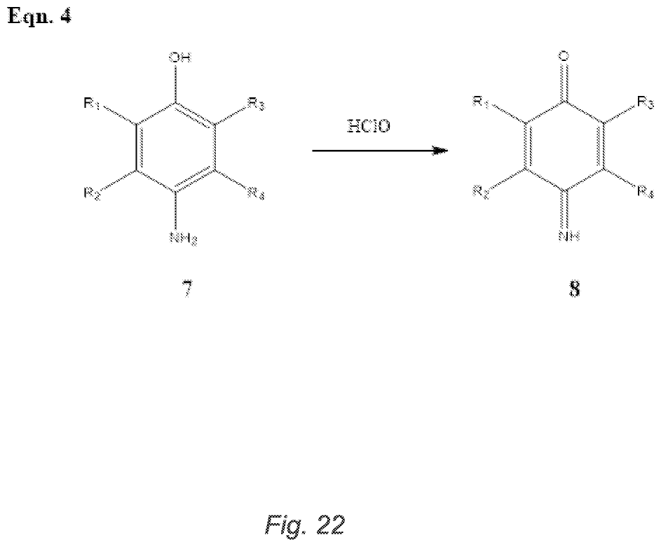

In another aspect, a method for making p-imino quinone via a dehydrogenation reaction as depicted in FIG. 22 is provided. The method includes a step of forming a biphasic mixture including an aqueous phase that includes a hypochlorite salt and an organic phase that includes a nonpolar solvent. Examples of hypochlorite salts include NaOCl, KOCl, LiOCl, and Ca(OCl)₂. A weak organic acid is added to the biphasic mixture to form hypochlorous acid. The mixture is agitated to promote the transfer of the hypochlorous acid from the aqueous phase to the organic phase. The aqueous phase is discarded. Therefore, the organic phase is collected. A p-aminophenol described by formula 7 is added to the organic phase to form the p-imino quinone described by formula 8. The mixture is agitated until an acidic aqueous phase is formed. Characteristically, the acidic aqueous phase includes hydrochloric acid (HCl). The acidic aqueous phase is maintained in this variation in order to recover the product as a hydrochloride. The nonpolar solvent is removed to recover the p-imino quinone described by formula 8:

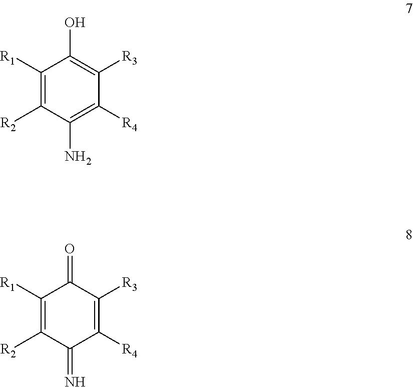

wherein R₁, R₂, R₃ and R₄ are each independently H, alkyl, or alkoxy. In a refinement, the molar ratio of the hypochlorite salt or the hypochlorous acid to the p-aminophenol described by formula 7 is about 1:1.

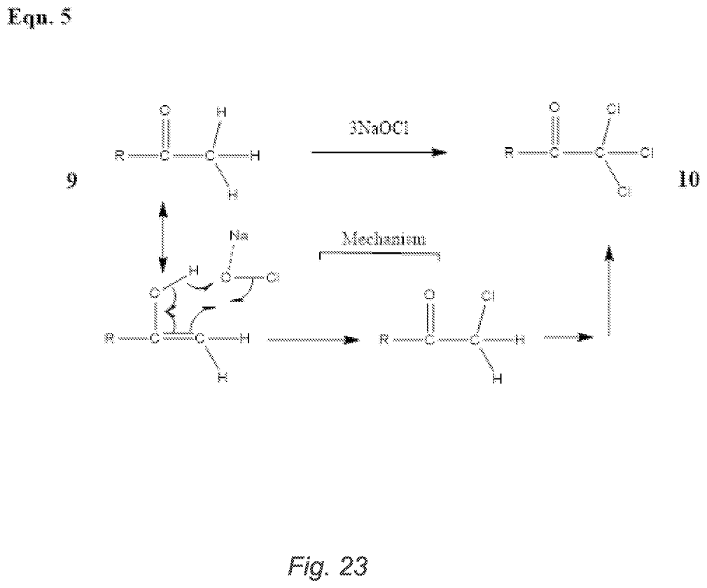

In another aspect, a method for making trichloromethyl ketones as depicted in FIG. 23 is provided. The method includes steps of reacting a ketone described by formula 9 with a hypochlorite salt to form a trichloromethyl ketone described by formula 10, and collecting the trichloromethyl ketone described by formula 10. Examples of hypochlorite salts include NaOCl, KOCl, LiOCl, and Ca(OCl)₂. In one refinement, if the ketone described by formula 9 is water soluble, the ketone described by formula 9 is mixed into an aqueous hypochlorite salt solution. In another refinement, the ketone described by formula 9 is water insoluble, the ketone described by formula 9 is mixed into a nonpolar solvent and combined with an aqueous hypochlorite salt solution to form a biphasic mixture, and the biphasic mixture is then agitated to provide a reaction for form the trichloromethyl ketone described by formula 10:

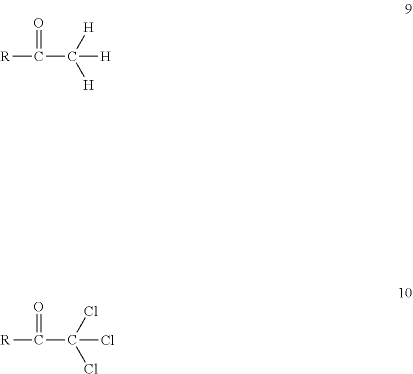

wherein R is linear or branched C₁₋₂₂ alkyl. In a refinement, R is methyl. In a refinement, the molar ratio of the hypochlorite salt to the ketone described by formula 9 is about 3:1.

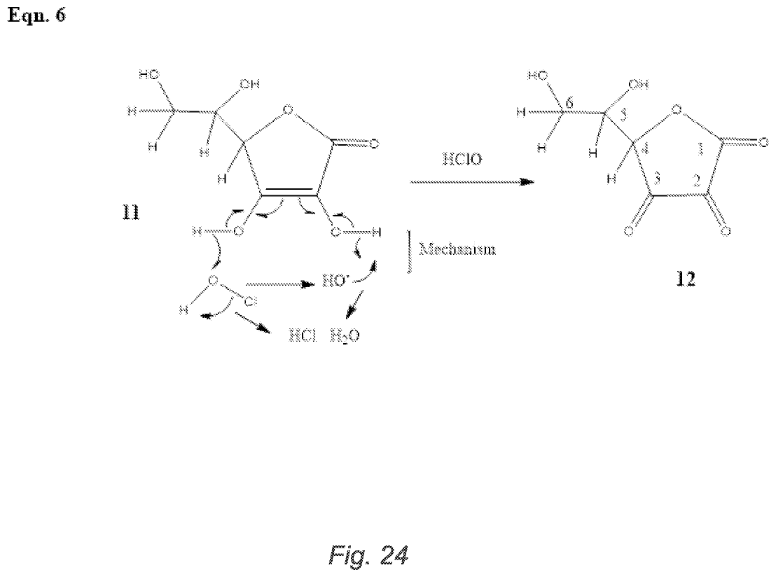

In another aspect, a method for making a dehydroascorbic acid via a dehydrogenation reaction as depicted in FIG. 24 is provided. The method includes a step of combining an aqueous solution that includes a hypochlorite salt with a nonpolar solvent to form a biphasic mixture including an organic phase and an aqueous phase. Examples of hypochlorite salts include NaOCl, KOCl, LiOCl, and Ca(OCl)₂. A weak organic acid is added to the biphasic mixture to form hypochlorous acid. The biphasic mixture is agitated such that the hypochlorous acid is transferred from the aqueous phase to the organic phase. The ascorbic acid described by formula 11 is added to the organic phase. The mixture is agitated until all of the solid ascorbic acid has reacted and is incorporated into the organic phase. A bottom layer that includes hydrochloric acid is discarded. The nonpolar solvent is removed to recover a compound described by formula 12:

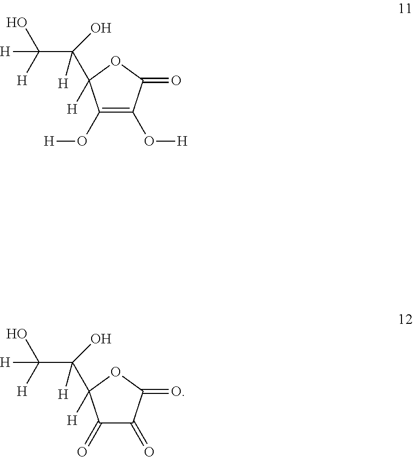 [SMILES: O=C1OC(C(O)CO)C(O)=C1O ; O=C1OC(C(O)CO)C(=O)C1=O]

In a refinement, the molar ratio of the hypochlorite salt or hypochlorous acid to the ascorbic acid described by formula 11 is about 1:1.

### SYNTHETIC METHODOLOGY AND EXAMPLES

### 1. General Methods Using Sodium Hypochlorite, Hypochlorous Acid or Potassium Hypoiodite

To a solution in water, or water/alcohol of the substrate (i.e., the compound to undergo the reaction) is added the number of equivalents of sodium hypochlorite required to produce the reaction. The number of equivalents of the hypochlorite are calculated from the balanced equation of the process based on the conditions of the dual radical system established above. After completion of the reaction, estimated by the disappearance of the starting material which can be observed using thin layer chromatography (TLC), the product is isolated by extraction or by concentration followed by extraction with a solvent of appropriate polarity. If hypochlorous acid is indicated for stability of the final product, as in the synthesis of dopaminochrome, the reactions above are run on a weak organic acid pre-acidified (e.g., pH 3 to 4) solution.

For compounds that are soluble in nonpolar solvents, a biphasic system of ethyl acetate/water is also appropriate. As such, the substrate is dissolved in the ethyl acetate with the mixture set above the water solution of sodium hypochlorite and shaken until completion (about 1-2 hours) as judged by TLC. The mixture can be separated using a separatory funnel and the organic phase is passed through a short silica gel or alumina plug and concentrated.

Typically, the reactions are carried out at ambient temperatures and pressure. For example, the reaction can be carried out at a temperature from 20 to 30° C. (e.g. room temperature) and a pressure of about 0.8 to 1.2 atm. It should be appreciated that the hypochlorite salts used in each of the methods can be replaced with other hypohalite salts such as potassium hypoiodite.

### 2. Cyclization Reactions

2.1 Synthesis of Indole: To a solution of phenylalanine in water are added two equivalents of sodium hypochlorite in water. After the evolution of carbon dioxide (CO₂) has subsided, the indole produced is isolated by extracting it with ethyl acetate. The purity of the material was verified by melting point, nuclear magnetic resonance (NMR) and mass spectroscopy (MS). Yield is 90%.

2.2 Synthesis of aminochromes: As depicted in FIG. 20, compounds known as catecholamines, represented by chemical structure 3 in Equation 2, whose examples include dopamine, adrenaline, and new structures obtained by variation of the groups R1 and R2, are converted in two consecutive steps with over 90% yield into compounds of structure 4, known generically as aminochromes, some of them involved in Alzheimer and Parkinson diseases. The method can include the following steps:
a. Placing a sodium hypochlorite solution (6%) in a separatory funnel and covering this aqueous solution with 6 times its volume of ethyl acetate (the organic phase)
b. Adding to the biphasic mixture above an equimolar amount of pure acetic acid. Care must be taken not to add an excess of the acid that would cause the production of chlorine gas.
c. Shaking the mixture above, with pauses for careful venting, to achieve complete transfer of the hypochlorous acid (HClO) formed in the aqueous phase into the organic phase.
d. Decanting and discarding from the separatory funnel the bottom aqueous phase.
e. Adding the solid catecholamine to the separatory funnel containing the HClO in the organic phase.
f. Shaking vigorously the heterogeneous mixture above until all the solid catecholamine is incorporated into the organic phase. The color of the solution will change from light yellow to the strong red color of the aminochrome.
g. Removing and recovering the ethyl acetate using a rotavap.
h. Collecting and weighing the solid aminochrome hydrochloride produced.

### 3. Dehydrogenation Reactions

3.1 Synthesis of 1,4-Benzoquinone: To a hydro/alcoholic solution of hydroquinone is added one equivalent of sodium hypochlorite in water. The mixture is stirred for 30 minutes, concentrated, and the solid produced extracted with alcohol, filtered through a plug of silica gel and concentrated to produce pure p-Quinone in 100% yield.

3.2 Synthesis of 1,4-Iminoquinone: As depicted in FIG. 22, compounds containing the p-aminophenol core, represented by chemical structure 7 in Equation 4, whose examples include the analgesic acetaminophen and new structures obtained by variation of the R groups are converted in a two-step, single batch process, with over 90% yield into compounds of structure 8, known generically as p-imino quinones, useful as antioxidants. The method can include the following steps:
a. Placing a sodium hypochlorite solution (6%) in a separatory funnel and covering this aqueous solution with 6 times its volume of ethyl acetate (the organic phase)
b. Adding to the biphasic mixture above an equimolar amount of pure acetic acid. Care must be taken not to add an excess of the acid that would cause the production of chlorine gas.
c. Shaking the mixture above, with pauses for careful venting, to achieve complete transfer of the hypochlorous acid (HClO) formed in the aqueous phase into the organic phase.
d. Decanting and discarding from the separatory funnel the bottom aqueous phase.
e. Adding an equimolar amount of the p-aminophenol to the separatory funnel containing the HClO in the organic phase.
f. Shaking the mixture above until a small aqueous phase is formed at the bottom of the separatory funnel. This aqueous phase contains hydrochloric acid (HCl) and it is maintained to produce the final compound as a hydrochloride.
g. Removing the ethyl acetate and water using a rotavap.
h. Collecting and weighing the p-imino quinone thereof produced.

3.3 Synthesis of 1,4-Iminoquinone: An ethyl acetate solution of p-aminophenol is suspended in water containing one equivalent of sodium hypochlorite. After approximately 2 hours and verification of reaction completion by TLC, a red 1,4-benzoquinone imine thereof can be isolated, passed through a plug of silica gel, and concentrated to produce 70% yield of pure material as verified by NMR and MS (mass calculated, M+H=108.0439, mass observed, M+H=108.0444).

3.4 Synthesis of dehydroascorbic acid: As depicted in FIG. 24, the compound known as ascorbic acid or vitamin C, represented by chemical structure 11 in Equation 6, is converted in a two-step, single batch process, with over 90% yield into a compound of structure 12, useful as a powerful antioxidant and known generically as dehydroascorbic acid, following the stepwise sequential diradical mechanism shown in Equation 6. Although the name dehydroascorbic acid has been mentioned in the scientific literature for many decades, the structure 12, a diketone, of Equation 6 has been dubbed impossible to make and never been detected (Ref. 1). Thus, the present method represents the only available way to prepare dehydroascorbic acid as represented by 12 in equation 6. The method can include the following steps:
a. Placing a sodium hypochlorite solution (6%) in a separatory funnel and covering this aqueous solution with 6 times its volume of ethyl acetate (the organic phase)
b. Adding to the biphasic mixture above an equimolar amount of pure acetic acid. Care must be taken not to add an excess of the acid that would cause the production of chlorine gas.
c. Shaking the mixture above, with pauses for careful venting, to achieve complete transfer of the hypochlorous acid (HClO) formed in the aqueous phase into the organic phase.
d. Decanting and discarding from the separatory funnel the bottom aqueous phase.
e. Adding an equimolar amount of the solid ascorbic acid to the separatory funnel containing the HClO in the organic phase.
f. Shaking the mixture above until all the solid is dissolved and a small aqueous phase containing hydrochloric acid (HCl) is formed at the bottom of the separatory funnel.
g. Decanting and discarding the acidic aqueous phase.
h. Filtering the organic solution through a solid plug containing a mixture of anhydrous sodium sulfate and sodium bicarbonate to remove leftover acid.
i. Removing and recovering the ethyl acetate using a rotavap.
j. Collecting and weighing the dehydroascorbic acid produced. The spectral values of the dehydroascorbic acid, never reported before, are as follows: MS(M-1): 173.0090 calc., 173.0092 found, IR: keto group: 1780 cm⁻¹ (strong), H-NMR(D₂O): 3.99 ppm (dd, 1H6), 4.09 ppm (dd, 1H5), 4.40 ppm (dd, 1H6), 4.58 ppm (s, 1H4). C13-NMR(D₂O): 173.25 ppm (C1), 105.33 ppm (C2), 91.00 ppm (C3), 87.26 ppm (C4), 75.90 ppm (C5), 72.57 ppm (C6).

3.5 Synthesis of p-quinones: As depicted in FIG. 19, compounds known generically as 5,7,8-trimethyl-6-chromanols, represented by chemical structure 1 in Equation 4, whose examples include the known α-tocopherol (a vitamin E), Trolox, and new structures by variation of the R substituent, are converted in a simple one-step procedure with over 90% yield into compounds of structure 2, shown in Equation 1, known generically as p-quinones, using sodium hypochlorite. By-products of the reaction are water and sodium chloride. Some of the p-quinones produced are known to have physiological actions from anticoagulants to anticancer agents, so other new variations are expected from the R substitution. The method includes the following steps:
a. Dissolving compound 1 in ethyl acetate and placing the solution in a separatory funnel.
b. Adding to the solution above an aqueous solution of sodium hypochlorite.
c. Shaking the biphasic mixture (Water/ethyl acetate) above vigorously with intervals for venting and observing at each interval the color of the organic (ethyl acetate) phase changing colors from colorless or lightly yellow to orange or red. The reaction is deemed complete when:
   1. The color of the organic phase does not change after repeated shakings, or, preferably,
   2. When TLC (Thin Layer Chromatography) using a mixture of Hexanes: Ethyl acetate (1:1) as the eluting solvent indicates complete absence of the starting material A.
d. Decanting from the separatory funnel the lower aqueous phase.
e. Washing 3 times with a small volume of water the organic phase in the separatory funnel with strong shaking.
f. Removing and recovering the organic phase using a rotavap.
g. Collecting and weighing the final product.

### 4. Hydroxylation Reactions

4.1 Synthesis of E-Quinone: In an aqueous alcoholic solution of α-tocopherol (a vitamin E), one equivalent of sodium hypochlorite in water was added. The mixture was stirred for 30 minutes and the E-quinone was extracted with ethyl acetate and concentrated to produce 100% yield of a reddish oil that gave MS of 447.3835 observed vs. MS of 447.3833 calculated. The carbon NMR in CDCl3 shows two peaks at 187.11 and 187.50 ppm corresponding to the ketone groups formed and absent in the α-tocopherol.

4.2 Synthesis of catechols: As depicted in FIG. 21, compounds containing the 7,8-dimethyl-6-chromanol core, represented by chemical structure 5 in Equation 3, whose examples include vitamin E isomers, and new structures obtained by variation of the R group are converted in a two-step, single batch process, with over 90% yield into compounds of structure 6, known generically as catechols, useful as powerful antioxidants. The method is also applicable to simple phenols that have a substitution in the para position. The method can include the following steps:
a. Placing a sodium hypochlorite solution (6%) in a separatory funnel and covering this aqueous solution with 6 times its volume of ethyl acetate (the organic phase)
b. Adding to the biphasic mixture above an equimolar amount of pure acetic acid. Care must be taken not to add an excess of the acid that would cause the production of chlorine gas.
c. Shaking the mixture above, with pauses for careful venting, to achieve complete transfer of the hypochlorous acid (HClO) formed in the aqueous phase into the organic phase.
d. Decanting and discarding from the separatory funnel the bottom aqueous phase.
e. Adding an equimolar amount of the chromanol to the separatory funnel containing the HClO in the organic phase.
f. Shaking the mixture above until a small aqueous phase is formed at the bottom of the separatory funnel. This aqueous phase contains hydrochloric acid (HCl).
g. Decanting and discarding the acidic aqueous phase at the bottom of the funnel.
h. Filtering the organic solution through a solid plug containing a mixture of anhydrous sodium sulfate and sodium bicarbonate to remove leftover acid.
i. Removing and recovering the ethyl acetate using a rotavap.
j. Collecting and weighing the catechol produced.

4.3 Synthesis of Trolox Quinone: Trolox and sodium hypochlorite in a 1:1 mixture in alcohol/water were stirred for 30 minutes. The yellow solution produced was concentrated, extracted with alcohol, and passed through a plug of silica gel to give, after concentration, 100% of Trolox Quinone as a yellow solid. The carbon NMR in DMSO shows two peaks at 186.73 and 187.38 ppm corresponding to the ketone groups formed and absent in the original Trolox compound.

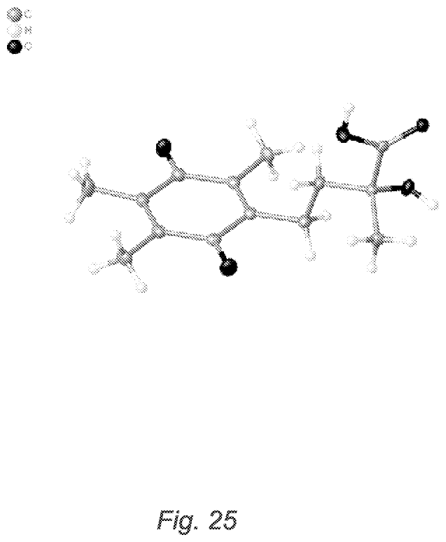

Crystal Data Trolo-Quinone: C₁₄H₁₈O₅ (M=266.28 g/mol): monoclinic, space group P2₁/c (no. 14), a=22.5724(14) Å, b=5.6734(4) Å, c=10.2326(6) Å, β=101.6500(10)°, V=1283.41(14) Å³, Z=4, T=100.00 K, β(Cu Kα)=0.870 mm⁻¹, Dcalc=1.378 g/cm³, 42133 reflections measured (7.998°≤2Θ≤158.64°), 2769 unique (Rᵢₙₜ=0.0257, R_{sigma}=0.0125) which were used in all calculations. The final R₁ was 0.0331 (I>2σ(I)) and wR₂ was 0.0894 (all data). FIG. 25 provided the chemical structure derived from the crystal data. (see FIG. 25).

### 5. Decarboxylation Reactions

5.1 Synthesis of 1-Pyrroline: A solution of proline in water was mixed with one equivalent of sodium hypochlorite in water and, after 5 minutes, the mixture was extracted with ethyl acetate, cooled to 5° C., passed through a plug of silica gel, and concentrated to produce 70% of the unstable 1-pyrroline. MS found of M+H=70.0652 vs. M+H calculated of 69.0651.

5.2 Synthesis of Indole: To a solution of phenylalanine in water, two equivalents of sodium hypochlorite in water were added. After the evolution of carbon dioxide (CO₂) had subsided, the indole produced was isolated by extracting with ethyl acetate. The purity of the indole was verified by melting point, nuclear magnetic resonance (NMR) and mass spectroscopy (MS). Yield was 90%.

5.3 Synthesis of 2,3,4,5-tetrahydropyridine: To a solution of lysine in water, two equivalents of sodium hypochlorite in water were added. After the evolution of carbon dioxide (CO2) had subsided, the 2,3,4,5-tetrahydropyridine produced was isolated by extracted with ethyl acetate. The purity of 2,3,4,5-tetrahydropyridine was verified by melting point, nuclear magnetic resonance (NMR) and mass spectroscopy (MS). Yield was 90%.

### 6. Halogenation Reactions

Synthesis of trichloromethyl ketones: As depicted in FIG. 23, methyl ketones, represented by chemical structure 9 in Equation 5, are converted in a single step, with yields from 25-90%, into compounds of structure 10, known generically as trichloromethyl ketones, following the stepwise sequential diradical mechanism shown in Equation 5. The trichloromethyl ketones are useful fungicides, insecticides, etc. This method avoids the use of chlorine gas in multistep syntheses and significantly reduces existing prices of these commodities. The method includes the following steps:
a. For water soluble ketones, such as acetone or butanone, mixing the ketones with a solution of sodium hypochlorite in a ratio 1 to 3 in a separatory funnel. The mixture will get hot and a layer of the trichloromethyl ketone will form slowly at the bottom of the funnel
b. Collecting in a timely fashion and weighing the trichloro-ketones by decanting them from the funnel. If the products are left in the funnel, further reactions will decrease their yield significantly.
c. For water insoluble ketones, such as acetophenone, mixing the ketones with ethyl acetate and shaken this organic solution with aqueous sodium hypochlorite, in a ratio 1 to 3, in a separatory funnel. The mixture will get hot and a layer of the trichloromethyl ketone will form slowly at the bottom of the funnel
d. Collecting in a timely fashion and weighing the trichloro-ketones by decanting them from the funnel. If the products are left in the funnel, further reactions will decrease their yield significantly.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A method comprising:
forming a biphasic mixture including an aqueous phase that includes a hypochlorite salt and an organic phase that includes a nonpolar solvent and a 5,7,8-trimethyl-6-chromanol described by formula 1;
agitating the biphasic mixture to allow a reaction between the hypochlorite salt and a 5,7,8-trimethyl-6-chromanol described by formula 1;
collecting the organic phase; and
removing the nonpolar solvent to recover a p-quinones described by formula 2: 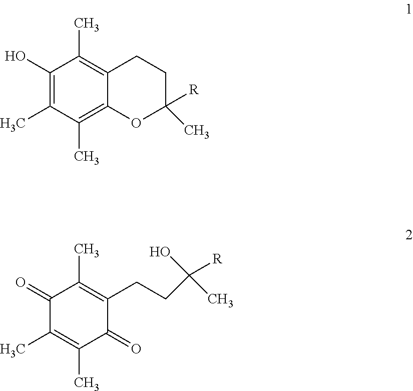
wherein R is selected from linear or branched C₁₋₂₂ alkyl, HC═O, or carboxy.

2. The method of claim 1, wherein the nonpolar solvent is an aprotic nonpolar solvent.

3. The method of claim 1, wherein the nonpolar solvent is ethyl acetate.

4. The method of claim 1, wherein the hypochlorite salt is selected from the group consisting of NaOCl, KOCl, LiOCl, and Ca(OCl)₂.

5. The method of claim 1, wherein the molar ratio of the hypochlorite salts to the 5,7,8-trimethyl-6-chromanol is about 1:1.

6. A method comprising:
combining an aqueous solution that includes a hypochlorite salt with a nonpolar solvent to form a biphasic mixture including an organic phase and an aqueous phase;
adding a weak organic acid to the biphasic mixture to form hypochlorous acid;
agitating the biphasic mixture such that the hypochlorous acid is transferred from the aqueous phase to the organic phase;
discarding the aqueous phase;
adding a catecholamine described by formula 3 to the organic phase that includes the hypochlorous acid; and
removing the nonpolar solvent to recover an aminochrome described by formula 4: 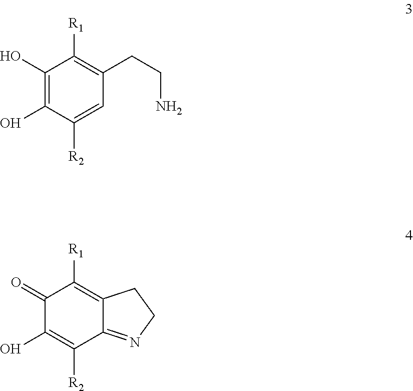
wherein:
R₁, R₂ are each independently H, alkyl, or alkoxy.

7. The method of claim 6, wherein the nonpolar solvent is an aprotic nonpolar solvent.

8. The method of claim 6, wherein the nonpolar solvent is ethyl acetate.

9. The method of claim 6, wherein the hypochlorite salt is selected from the group consisting of NaOCl, KOCl, LiOCl, and Ca(OCl)₂.

10. The method of claim 6, wherein the molar ratio of the hypochlorite salt or the hypochlorous acid to the catecholamine described by formula 3 is about 1:1.

11. A method for making catechols comprising:
forming a biphasic mixture including an aqueous phase that includes a hypochlorite salt and an organic phase that includes a nonpolar solvent;
adding a weak organic acid to the biphasic mixture to form hypochlorous acid in the aqueous phase;
agitating the biphasic mixture to transfer the hypochlorous acid formed in the aqueous phase into the organic phase;
discarding the aqueous phase;
adding a chromanol described by formula 5 to the organic phase;
agitating the organic phase until an acidic aqueous phase is formed, the acidic aqueous phase including hydrochloric acid (HCl);
discarding the acidic aqueous phase;
removing the nonpolar solvent; and
collecting a catechol described by formula 6: 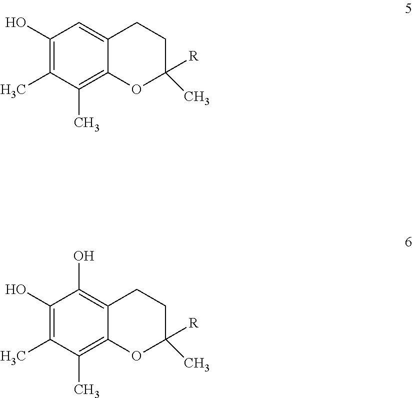
wherein R is selected from linear or branched C₁₋₂₂ alkyl, HC═O, or carboxy. solvent.

12. The method of claim 11, wherein the nonpolar solvent is an aprotic nonpolar

13. The method of claim 11, wherein the nonpolar solvent is ethyl acetate.

14. The method of claim 11, wherein the hypochlorite salt is selected from the group consisting of NaOCl, KOCl, LiOCl, and Ca(OCl)₂.

15. The method of claim 11, wherein the molar ratio of the hypochlorite salt or the hypochlorous acid to the chromanol described by formula 5 is 1:1.

16. A method comprising:
forming a biphasic mixture including an aqueous phase that includes a hypochlorite salt and an organic phase that includes a nonpolar solvent;
adding a weak organic acid to the biphasic mixture to form hypochlorous acid;
agitating biphasic mixture such that the hypochlorous acid is transferred from the aqueous phase to the organic phase;
discarding the aqueous phase;
adding a p-aminophenol described by formula 7 to the organic phase to form a p-imino quinone described by formula 8; and
removing the nonpolar solvent and any water therein to recover the p-imino quinone described by formula 8 thereof: 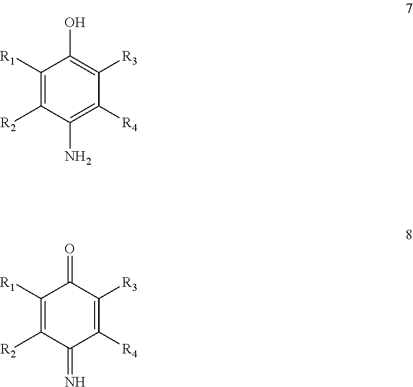
wherein R₁, R₂, R₃ and R₄ are each independently H, alkyl, or alkoxy. solvent.

17. The method of claim 16, wherein the nonpolar solvent is an aprotic nonpolar

18. The method of claim 16, wherein the nonpolar solvent is ethyl acetate.

19. The method of claim 16, wherein the hypochlorite salt is selected from the group consisting of NaOCl, KOCl, LiOCl, and Ca(OCl)₂.

20. The method of claim 19, wherein the molar ratio of the hypochlorite salt or the hypochlorous acid to the p-aminophenol described by formula 7 is about 1:1.

21. A method comprising:
reacting a ketone described by formula 9 with a hypochlorite salt to form a trichloromethyl ketone described by formula 10; and
collecting the trichloromethyl ketone described by formula 10, wherein
if the ketone described by formula 9 is water soluble, the ketone described by formula 9 is mixed into an aqueous hypochlorite salt solution; and
if the ketone described by formula 9 is water insoluble, the ketone described by formula 9 is mixed into a nonpolar solvent and combined with an aqueous hypochlorite salt solution to form a biphasic mixture, the biphasic mixture is then agitated to provide a reaction for form the trichloromethyl ketone described by formula 10: 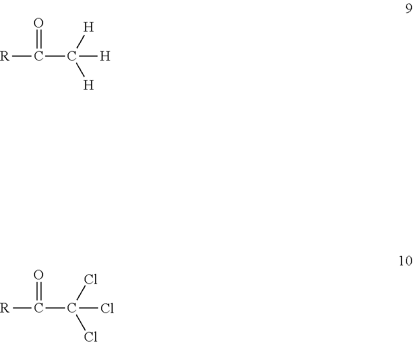
wherein R is linear or branched C₁₋₂₂ alkyl.

22. The method of claim 21, wherein the nonpolar solvent is an aprotic nonpolar solvent.

23. The method of claim 21, wherein the nonpolar solvent is ethyl acetate.

24. The method of claim 21, wherein the hypochlorite salt is selected from the group consisting of NaOCl, KOCl, LiOCl, and Ca(OCl)₂.

25. The method of claim 21, wherein the molar ratio of the hypochlorite salt to the ketone described by formula 9 is 3:1.

26. A method comprising:
combining an aqueous solution that includes a hypochlorite salt with a nonpolar solvent to form a biphasic mixture including an organic phase and an aqueous phase;
adding a weak organic acid to the biphasic mixture to form hypochlorous acid;
agitating the biphasic mixture to transfer the hypochlorous acid from the aqueous phase to the organic phase;
adding an ascorbic acid described by formula 11 to the organic phase;
agitating the mixture until all of the solid ascorbic acid has reacted and is incorporated into the organic phase;
discarding a bottom layer that includes hydrochloric acid; and
removing the nonpolar solvent to recover a compound described by formula 12:

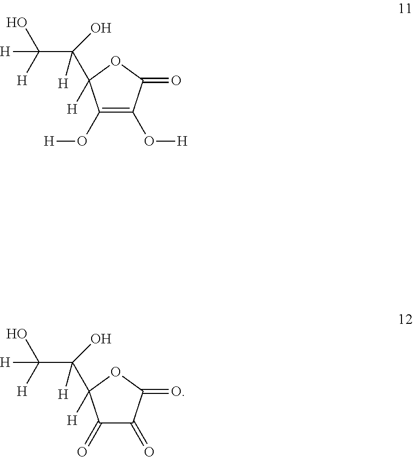 [SMILES: O=C1OC(C(O)CO)C(O)=C1O ; O=C1OC(C(O)CO)C(=O)C1=O]

27. The method of claim 26, wherein the nonpolar solvent is an aprotic nonpolar solvent.

28. The method of claim 26, wherein the nonpolar solvent is ethyl acetate.

29. The method of claim 26, wherein the hypochlorite salt is selected from the group consisting of NaOCl, KOCl, LiOCl, and Ca(OCl)₂.

30. The method of claim 26, wherein the molar ratio of the hypochlorite salt or hypochlorous acid to the ascorbic acid described by formula 11 is 1:1.
